(19) United States
(12) Patent Application Publication
(10) Pub. No.: US 2004/0029254 A1
(43) Pub. Date: Feb. 12, 2004

(54) Method of screening anti-bacterial agents for effectiveness in treating persistant intracellular infections

(75) Inventors: Walter E. Stamm, Seattle, WA (US); Robert J. Suchland, Seattle, WA (US)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(73) Assignee: University of Washington

(21) Appl. No.: 10/438,287

(22) Filed: May 13, 2003

Related U.S. Application Data
(60) Provisional application No. 60/380,896, filed on May 15, 2002.

(51) Int. Cl. C12N 1/20

(52) U.S. Cl. 435/252.3

(57) ABSTRACT

The present application provides methods of screening anti-bacterial agents for effectiveness in treating persistent intracellular infection by bacteria capable of forming intracytoplasmic inclusions in cells.

### CROSS-REFERENCE TO RELATED APPLICATION

This application claims the benefit of U.S. Provisional Application No. 60/380,896, filed May 15, 2002.

### GOVERNMENT RIGHTS

This invention was made with government support under grant number AI-48769 awarded by the National Institutes of Health. The government has certain rights in the invention.

### FIELD OF THE INVENTION

This invention relates to methods of screening anti-bacterial agents for effectiveness in treating persistent intracellular infections.

### BACKGROUND OF THE INVENTION

This invention relates to the use of a novel method of screening anti-bacterial agents for effectiveness in the treatment of persistent intracellular infections by bacteria that form intracytoplasmic inclusions.

Chlamydiae are obligate intracellular microorganisms which parasitize eukaryotic cells. They lack several metabolic and biosynthetic pathways and depend on the host cell for intermediates including ATP. Chlamydiae have a unique biphasic developmental cycle with distinct morphological and functional forms. This developmental growth cycle alternates between (1) intracellular life forms, of which two are currently recognized, a metabolically-active, replicating organism known as the reticulate body (RB) and a persistent, non-replicating organism known as the cryptic phase; and (2) an extracellular life form that is an infectious, metabolically-inactive form known as the elementary body (EB). EBs are small (300-400 nm), infectious, spore-like forms which are metabolically inactive, non-replicating, and found most often in the acellular milieu. EBs possess a rigid outer membrane which protects them from a variety of physical insults such as enzymatic degradation, sonication and osmotic pressure.

A complex series of events occurs during the developmental cycle of chlamydiae. The infectious EB attaches to a columnar epithelial cell and, through parasite specified endocytosis, enters the cell within a cytoplasmic vacuole known as a phagosome. Once inside the phagosome, the EB undergoes changes in its cell envelope whereby it loses its rigidity and reorganizes into the more flexible and less dense structure of the RB. The RB grows and then replicates by binary fission with a doubling time of two to three hours. Replication continues until the host cell cytoplasm is almost filled with chlamydiae. These RBs contain no cell wall and are detected as inclusion bodies. After division the RBs synthesize an outer cell wall and reorganize into EBs that are released from the cell as infectious particles, thereby allowing the cycle to begin again.

There are other Chlamydia-like bacteria that have biphasic developmental cycles with distinct morphological and functional forms including EBs and RBs as described above. These bacteria include Simkania negevensis and Parachlamydia acanthamoeba. Both of these bacteria are also obligate intracellular parasites, which can exist in two morphologic forms (RB and EB) and can be detected by intracellular inclusions (Everett et al., Int. J. Syst. Bacteriol. 49: 415-440, 1999).

Species of chlamydiae that cause human diseases include Chlamydia (C.) trachomatis, C. pneumoniae, and C. psittaci. C. trachomatis is transmitted from person-to-person and is the causative agent of trachoma (a leading cause of blindness), inclusion conjunctivitis, sexually transmitted genital and rectal infections, salpingitis, proctitis, epididymitis, lymphogranuloma venereum, and infant pneumonia. C. pneumoniae is also transmitted from person-to-person and is the causative agent of a typical pneumonia (walking pneumonia), pharyngitis, bronchitis, sinusitis, and atherosclerosis. C. psittaci is typically transmitted through contact with an infected bird or bird droppings and is the causative agent of some upper respiratory infections and pneumonitis.

Infection by chlamydiae induces a significant immune response at the cellular level. For example, genital lesions produced by C. trachomatis frequently elicit a vigorous influx of lymphocytes, macrophages, and plasma cells, suggesting the development of humoral and cellular immunity. Yet, clinically, the initial infection is frequently varied in symptomatology and may even be asymptomatic. This may be due to the protected intracellular habitat in which chlamydiae exist allowing them to persist in the face of an immune response.

Once fully established, the chlamydiae are difficult to eradicate, with frequent relapse following antibiotic therapy. Evidence also indicates that the chlamydiae may become dormant and are then shed in quantities too few to reliably detect by culture. For example, C. trachomatis has been repeatedly cultured from some patients despite anti-bacterial therapy (Keshishyan, H., et al., Nature 244:173-174, 1973; Somani, J., et al., J. Infect. Dis. 181:1421-1427, 2000, Jones, R. B., J. Infect. Dis. 162:1309-1315, 1990). In addition, C. pneumoniae is well-accepted as a human pathogen that may be difficult to eradicate by standard antibiotic therapy (Hammerschlag et al., Clin. Infect. Dis. 14:178-182, 1992). C. pneumoniae is known to persist as a silent or mildly symptomatic pathogen, resulting in a chronic, persistent. infection (Schacter, J. In: Baun, A. L., eds. Microbiology of Chlamydia, Boca Raton, Fla., CRC Press, 1988, pp. 153-165). In vitro studies on the persistence of chlamydiae despite specific and appropriate antibiotic therapy, have suggested that the presence of antibiotics promotes the formation of an intracellular, non-replicative state typically referred to as the latent or cryptic phase (Beatty et al., Microbiol. Rev. 58:686-699,1994). Removal of the antibiotic allows the organism to resume replication. Thus, in this way, the organism can escape current antibiotic therapy used in clinical practice.

In the United States alone, an estimated three million individuals contract Chlamydia each year. The estimated annual cost of Chlamydia and its complications is approximately two billion dollars. The ability of this bacteria to remain in a chronic, persistent form, despite the use of standard anti-bacterial therapies, suggests that the long-term costs of chronic diseases associated with chlamydial infections may be even greater.

In view of the chronic and persistent nature of Chlamydia and Chlamydia-like bacterial infections, there is a need for a reliable and effective way to identify anti-bacterial agents that can totally eradicate these bacteria, thereby preventing the long-term sequelae of such persistent infections.

### SUMMARY OF THE INVENTION

The present invention features a unique method for screening both known and novel anti-bacterial agents for their effectiveness in treating persistent intracellular infections by Chlamydia and Chlamydia-like bacteria.

Accordingly, in a first aspect the invention features a method of screening an anti-bacterial agent for its effectiveness in treating a persistent intracellular infection by bacteria capable of forming intracytoplasmic inclusions in cells. In this method, a first culture of cells is inoculated with bacteria and then contacted with an anti-bacterial agent for 24 to 72 hours. The number of intracytoplasmic inclusions that are altered in size and morphology in this culture of cells is determined relative to a culture of cells inoculated with the same bacteria but not contacted with the anti-bacterial agent. The number of altered inclusions at various concentrations of anti-bacterial agent is then used to calculate the minimal inhibitory concentration (MIC) of the anti-bacterial agent being screened. A second culture of the same cells is also inoculated with the same bacteria and then contacted with the same anti-bacterial agent for 24 to 72 hours. This second culture of cells is passaged three times with continued anti-bacterial treatment as described above, and the number of morphologically normal intracytoplasmic inclusions is determined relative to a culture of cells inoculated with the same bacteria but not contacted with the anti-bacterial agent. The number of morphologically normal intracytoplasmic inclusions is then used to calculate the minimal chlamydicidal concentration after three passages (MCC₃) of the anti-bacterial agent being screened. Finally, the MCC₃/MIC ratio for the test anti-bacterial agent is determined and an anti-bacterial agent having an MCC₃/MIC ratio of 100 or less is considered to be effective for treating persistent intracellular infections by bacteria that form intracytoplasmic inclusions in cells.

There are particular cell culture-bacterial species pairings which compose preferred embodiments of the first aspect. In one particular embodiment, the first and second cultures of cells are of McCoy cells and the intracellular bacteria is C. trachomatis. In another preferred embodiment, the first and second cultures of cells are of McCoy cells and the intracellular bacteria is C. psittaci. In yet another preferred embodiment, the first and second cultures of cells are of HEp-2 cells and the intracellular bacteria is C. pneumoniae.

The invention also features preferred methods for the inoculation of the cell cultures with the bacteria and the contacting of the inoculated cells with the anti-bacterial agent. For example, in a preferred embodiment, the cells are inoculated at a multiplicity of infection (MOI) of 0.2-2.0 IFU/cell. This MOI generally results in infection of about 50% of the cells. In another preferred embodiment, the addition of anti-bacterial agent occurs within 0 to 8 hours of inoculation.

The method featured in the present invention includes the steps of determining the number of inclusions, in the first culture of cells, that are altered in size and morphology, and determining the number of inclusions in the second culture of cells that are morphologically normal. Several types of staining may be used to detect the intracytoplasmic inclusions, including, but not limited to immunofluorescence detection, Giemsa staining, and iodine staining. In a preferred embodiment, immunofluorescence detection is used to determine the number of intracytoplasmic inclusions that are altered in size and morphology. In a related preferred embodiment, immunofluorescence detection is performed using a genus-specific antibody. One example of such an antibody is CF-2.

In a preferred embodiment of the first aspect, the intracellular bacteria are a species of Chlamydia or Chlamydia-like bacteria. Species of Chlamydia and Chlamydia-like bacteria include but are not limited to C. trachomatis, C. pneumoniae, C. psittaci, C. suis, Simkania negevensis, and Parachlamydia acanthamoeba. Cell lines used for propagating the bacterial culture include but are not limited to HeLa, McCoy, BGMK, Hep-2, HL, and Vero cells.

For the purpose of the present invention, the following abbreviations and terms are defined below.

By “altered in size and morphology” is meant inclusions that are generally smaller or larger, and irregular in size or shape as compared to morphologically normal inclusions (see definition below).

By “anti-bacterial agent” is meant any agent, which is or may be used in the treatment of microbial infections. Known anti-bacterial agents include but are not limited to rifamycins, azalides, ketolides, streptogramins, ampicillin, amoxicillin, quilolones, fluoroquinolones, sulfonamides, isonicotinic congeners, and tetracyclines.

By “bacteria” is meant a unicellular prokaryotic microorganism that usually multiplies by cell division.

By “contacting” is meant adding or overlaying, e.g., an anti-bacterial agent into or upon a cell culture media.

By “inoculating” is meant implanting or overlaying, e.g., infectious bacteria into or upon a cell culture media.

By “intracytoplasmic inclusion” is meant a replicating reticulate body (RB) that has no cell wall. Such inclusions may be detected, for example, through chlamydiae sample isolation and propagation on a mammalian cell lines, followed by fixing and staining using one of a variety of staining methods including Giemsa staining, iodine staining, and immunofluorescence. These inclusions have a typical round or oval appearance.

By “minimal chlamydicidal concentration (MCC)” is meant the lowest concentration of anti-bacterial agent that produced no morphologically normal inclusions. The MCC is determined using microtiter plates for cell growth and by passaging cells one time using a freeze-thaw method.

By “minimal chlamydicidal concentration 1 (MCC₁)” is meant the lowest concentration of anti-bacterial agent that produced no morphologically normal inclusions using glass shell vials for cell growth and by passaging cells one time.

By “minimal chlamydicidal concentration 3 (MCC₃)” is meant the lowest concentration of anti-bacterial agent that produced no morphologically normal inclusions using glass shell vials for cell growth and by passaging cells three times.

By “minimal inhibitory concentration (MIC)” is meant the concentration of anti-bacterial agent that is one 2-fold dilution greater that the MIC₉₀. By MIC₉₀ is meant the concentration of anti-bacterial agent in which 90% or more of the inclusions are altered in size and morphology.

By “morphologically normal” is meant inclusions that are oval-shaped with smooth walls. Morphologically normal inclusions consume more than 25% of the cell cytoplasm.

By “persistent infection” is meant an infection that is not completely eradicated through standard treatment regimens using anti-bacterial agents. Persistent infections may be classified as such by culturing the bacteria from a patient and demonstrating repeated anti-bacterial resistance in vitro or by determination of anti-bacterial treatment failure in a patient. As used herein, a persistent infection in a patient includes any recurrence of chlamydial infection, after receiving anti-bacterial treatment, from the same species (e.g., C. trachomatis) more than two times over the period of two or more years. In addition an in vivo persistent infection can be identified through the use of a reverse transcriptase polymerase chain reaction (RT-PCR) to demonstrate the presence of 16S rRNA transcripts in bacterially infected cells after treatment with anti-bacterial agents (Dresses-Werringloer, U., et al., Antimicrob. Agents Chemother. 12:3288-3297, 2000).

All patent applications and publications mentioned in this specification are herein incorporated by reference to the same extent as if each independent patent application and publication was specifically and individually indicated to be incorporated by reference.

### DETAILED DESCRIPTION OF THE PREFERRED EMBODIMENT

The present invention satisfies an existing need for methods to screen anti-bacterial compounds for efficacy in treatment of persistent intracellular infections by bacteria that form intracytoplasmic inclusions in cells. The invention is based on the discovery that determination of MIC and MCC₃ values for any anti-bacterial as well as determination of the MCC₃ to MIC ratio can be used as a quantitative method to determine the potential therapeutic effectiveness of an anti-bacterial in treatment of persistent bacterial infections.

In addition, the invention features the use of specific cell line-bacteria strain pairing for optimal testing and consistency in anti-bacterial screening. These cell lines include but are not limited to McCoy, HeLa, BGMK, HEp-2, HL, and Vero cells. McCoy, HeLa, HEp-2, HL, and Vero can be obtained from American Type Culture Collection (ATCC, Manassas, Va., catalog numbers CRL-1696, CCL-2, CCL-23, CCL-240, and CCL-81, respectively). BGMK cells can be obtained from Bio-Whittaker Inc. (Walkersville, Md.; catalog number 71-176).

Optimal cell line-species pairing includes but is not limited to McCoy cells with C. trachomatis, McCoy cells with C. psittaci, and HEp-2 cells with C. pneumoniae.

The present invention features the determination of the MCC₃/MIC ratio for anti-bacterial agents. The significance of this ratio is that it allows for a quantitative evaluation of the effectiveness of an anti-bacterial agent in treating persistent intracellular bacterial infections. Determination of MIC alone may be sufficient to establish the amount of anti-bacterial agent required to eradicate an acute infection, but as is demonstrated in the following examples, it is not reflective of the effectiveness of the anti-bacterial in the treatment of persistent infections.

The following examples describe the assays used in the present invention and the results obtained for various anti-bacterial agents screened against several species of chlamydiae. The examples refer specifically to chlamydiae but it will be understood by those skilled in the art that the equivalent assays can be performed on other obligate intracellular that form inclusions in the cell.

### EXAMPLES

Methods

The following methods were used for the examples described below.

Organisms

Laboratory reference strains for C. trachomatis (serovars B, D, E, F, G, H, I, Ia, H, J, K, and L2), C. pneumoniae (TW-183 and CWL-029), and C. psittaci (6BC and GPIC) are maintained and routinely used in our laboratory for research. The tetracycline-resistant C. suis strain R-19 was provided by Arthur A. Andersen, National Animal Disease Center, Ames, Iowa. Clinical strains were selected from a large archive of serotyped clinical isolates obtained from patients who had a culture-documented C. trachomatis genital infection at any of the Seattle-King County Health Department Sexually Transmitted Disease clinics. Patients with single C. trachomatis isolates were defined as patients who had no record in our database of having an additional previous or subsequent chlamydial infection. Persistent isolates were chosen from patients who had 3 or more same-serovar recurrent C. trachomatis infections over a period of greater than 2 years with no intervening infections of a different chlamydial serovar. Treatment-failure isolates were selected from patients who had 2 or more visits with a positive C. trachomatis culture after receiving documented treatment. Four unknown strains without identifiers were provided by the Centers for Disease Control and Prevention (CDC) for susceptibility testing; two isolates were from patients diagnosed as treatment failures and had been determined to be multi-drug resistant by CDC Laboratories, and two isolates were controls without anti-bacterial resistance.

Anti-Bacterial Susceptibility Testing

Anti-bacterial susceptibility testing of C. trachomatis, C. pneumoniae, and C. psittaci strains in six cell lines (HeLa, McCoy, BGMK, HEp-2, HL and Vero) without passage (MIC) on 96-well microtiter plates and by multiple passage (MCC) in 48-well microtiter plates and 12 mm glass shell vials was undertaken with doxycyline, azithromycin, erythromycin, ofloxacin, and tetracycline. Anti-bacterial susceptibility testing in 48-well microtiter plates and 12 mm glass shell vials for the C. suis strain R-19 in McCoy cells, the reference C. trachomatis and C. psittaci strains in McCoy cells, the reference C. pneumoniae strains in HEp-2 cells, and the clinical isolates of C. trachomatis in McCoy cells was undertaken with doxycycline, azithromycin, and ofloxacin. Cells were maintained in antimicrobial-free growth medium, consisting of minimal essential medium with 10% fetal bovine serum and 220 μg/L L-glutamine added. The inoculum size of infectious chlamydial forms for all MIC/MCC comparisons was 10,000-50,000 inclusion-forming units (IFU)/well per 48-well microtiter plate or on 12 mm coverslips in shell vials resulting in infection of approximately 10-50% of cells in the monolayers.

MIC determinations were performed on monolayers in 96-well plates. For all centrifugation procedures, a Beckman model J-6M centrifuge was utilized. Each chlamydial strain was inoculated into three rows of twelve wells, centrifuged at 1200×g for 1 h at 37° C. and then supernatants were aspirated. Anti-bacterial agents were obtained in powder form, weighed and adjusted for purity, and reconstituted according to manufacturer's instructions. Dilution schemes were prepared using 2-fold dilutions in anti-bacterial-free growth medium containing cycloheximide (1.0 μg/ml). 100 μl of each dilution for each anti-bacterial was added to the appropriate wells to give a final concentration range of 0.008-128 μg/ml. Passage of tissue culture plates was done by a freeze-thaw method where monolayers were frozen at −70° C. thawed in a 37° C. water bath, disrupted with a pipette tip, passed onto fresh monolayers, and centrifuged at 1200×g for 1 h at 37° C. Wells were then aspirated and overlaid with appropriate 2-fold drug dilutions made in anti-bacterial-free growth medium with cycloheximide added. Cells were incubated at 37° C. in 4% C02 for 24-72 h (48 h for C. trachomatis, 72 h for C. pneumoniae, and 24 h for C. psittaci) and fixed with methanol. Chlamydial inclusions were detected by fluorescence using a genus-specific monoclonal antibody CF-2 (Washington Research Foundation, Seattle).

Passage of monolayers in shell vials differed from culture plates in that after incubation, medium containing anti-bacterial was aspirated and cells were rinsed twice with Hanks Balanced Salt Solution. Cells were then disrupted by sonication using an ultrasonic processor and the resulting suspension was centrifuged at 300×g for 10 min at 4° C. to remove debris. The supernatant was inoculated onto fresh monolayers in shell vials, centrifuged at 1200×g for 1 h at 37° C., aspirated, and overlaid and incubated as described previously. For each passage, 100 μl of each passage vial was added to corresponding wells on a 96-well microtiter plate for observation of inclusions. A laboratory reference strain, serovar D/uw-3/cx, which is anti-bacterial sensitive, was used in all comparisons and in MIC/MCC/MCC₃ comparison experiments.

The MIC₉₀ was defined as the concentration of drug in which 90% or more of inclusions are altered in size and morphology. The MIC was defined as the concentration of drug that is one 2-fold dilution less than the MIC₉₀. The MCC was designated as the lowest concentration of drug that produced no morphologically normal inclusions by one freeze-thaw passage in microtiter plates (microtiter plate method) or by one passage in shell vials (shell vial method) in anti-bacterial-free medium. The MCC₃ was determined to be the MCC after three passages in shell vials (shell vial method).

To determine cell line variation on MCC₃ levels, several representative chlamydiae strains were passaged three times in McCoy, BGMK, HeLa and HEp-2 cells. To test for selection of anti-bacterial-resistant organisms, chlamydiae surviving the highest anti-bacterial concentration after three passages in shell vials were retested against the respective anti-bacterial and the MIC was determined.

A 2-fold dilution scheme from 320,000 to 300 IFU/well (48-well microtiter plate) inoculum of C. trachomatis strain UW-3 (serovar D) was used to determine effect of inoculum size on MIC, MCC and MCC₃ levels. The effect that time elapsed between infection and addition of a given anti-bacterial had on MIC values was tested by adding drug at 2-hour intervals up to 24 hours after infection.

### Example 1

### Anti-Bacterial susceptibility Testing of Chlamydia Species in Different Cell Lines

In order to test the importance of host cell line type when screening anti-bacterials, six different cell lines were used with various chlamydial species for anti-bacterial susceptibility. Cell lines included McCoy, HeLa, BGMK, HEp-2, HL, and Vero. MIC, MCC, and MCC₃ values were calculated for each chlamydiae species in each cell line. The MIC values for doxycycline and ofloxacin were comparable in all cell lines for C. trachomatis, C. pneumoniae, and C. psittaci strains tested. Interestingly, there was considerable MIC variation when C. trachomatis strains were tested against azithromycin and erythromycin with isolates tested in HeLa and HL cell lines having the lowest MIC values and those tested in the BGMK cell line having significantly higher MIC values (Table 1). These results demonstrate the importance of the cell line selection for inoculation with the intracellular bacteria; specific cell-line organism pairings are suggested herein.

TABLE 1
EFFECT OF CELL LINE ON MIC, MCC, AND MCC₃ FOR CHLAMYDIA TRACHOMATIS SEROVAR D^{A}

| Cell Type | Doxycycline | | | Azithromycin | | | Erythromycin | | | Ofloxacin | | | Tetracycline | | |
|---|---|---|---|---|---|---|---|---|---|---|---|---|---|---|---|
| (origin) | MIC | MCC | MCC₃ | MIC | MCC | MCC₃ | MIC | MCC | MCC₃ | MIC | MCC | MCC₃ | MIC | MCC | MCC₃ |
| McCoy | 0.064 | >8 | 64 | 0.125 | >8 | 64 | 0.25 | >8 | 128 | 1 | >8 | 128 | 0.25 | >8 | 64 |
| (mouse) | | | | | | | | | | | | | | | |
| HeLa | 0.064 | 1 | 4 | 0.016 | 1 | 8 | 0.032 | 2 | 4 | 1 | 4 | 16 | 0.25 | 2 | 16 |
| (human) | | | | | | | | | | | | | | | |
| BGMK | 0.064 | >8 | 64 | 1.0 | >8 | 64 | 2 | >8 | 128 | 1 | >8 | 128 | 0.25 | >8 | 64 |
| (simian) | | | | | | | | | | | | | | | |
| HEp-2 | 0.064 | 1 | 4 | 0.064 | 4 | 32 | 0.125 | 4 | 8 | 1 | 4 | 64 | 0.25 | 4 | 32 |
| (human) | | | | | | | | | | | | | | | |
| HL | 0.064 | 2 | 32 | 0.008 | 4 | 64 | 0.016 | 4 | 0.5 | 1 | 8 | 64 | 0.25 | 4 | 32 |
| (human) | | | | | | | | | | | | | | | |
| Vero | 0.064 | 2 | 8 | 0.25 | 2 | 4 | 0.5 | 4 | 16 | 1 | 8 | 32 | 0.25 | 2 | 16 |
| (simian) | | | | | | | | | | | | | | | |

^{A}All concentration values are given in μg/ml. MIC, minimal inhibitory concentration determined as one 2-fold dilution concentration more that the MIC₉₀ (dilution in which 90% or more of inclusions are altered in size and morphology); MCC, minimal chlamydicidal concentration following one freeze-thaw passage in microtiter plates; MCC₃ minimal chlamydicidal concentration following three passages in shell vials.

### Example 2

### Effect of Inoculum Size and Timing of Anti-Bacterial Treatment on susceptibility Testing in C. trachomatis

A range of inoculum sizes was tested in order to determine the optimal chlamydiae inoculum size for anti-bacterial screening. There was no observed difference in MIC values when the inoculum size of viable Chlamydia was varied over the range of 300 to 300,000 IFU/well. When an inoculum of less than 300 IFU/well was used, it was difficult to find a sufficient number of inclusions to read accurately. Variation of inoculum size did influence MCC values by shell vial passage, with elevation of MCC and MCC₃ levels above that of the MIC (Table 2). However, when the inoculum fell below 5000 IFU/well, there was no observed difference between MIC and MCC values. This suggests that the survival rate of antibiotic exposed Chlamydia is somewhere around one out of every 5000 infected EBs.

Anti-bacterial agents were added at various time points after inoculation in order to test the relevance of time between infection and addition of anti-bacterial agents on anti-bacterial susceptibility testing in C. trachomatis. There were no observable differences in MIC values when an anti-bacterial agent was added at 2-hour intervals from 0 to 8 hours after infection, but the MIC increased steadily after 8 hours, suggesting that the drug had a weakened effect on chlamydial development after 8 hours post-infection

These results indicate that there is variation in MIC and MCC₃ determination depending on the inoculum size used and the time of treatment with anti-bacterial agent. The present invention provides a specific range for multiplicity of infection used when screening anti-bacterial agents (0.2-2.0 IFU/cell), which results in infection of approximately 50% of the cells. In addition, the invention provides a range of times (0 to 8 hours after the inoculation) for contacting the cells with the anti-bacterial agent. These ranges limit the variability in the results obtained by the method of screening described herein.

TABLE 2
EFFECT OF INOCULUM SIZE ON DOXYCYCLINE MIC, MCC,
AND MCC₃ FOR C. TRACHOMATIS
SEROVAR D^{A}.

| Inoculum | | | |
|---|---|---|---|
| (IFU/well × 10³) | MIC | MCC | MCC₃ |
| 320 | 0.064 | >8 | 64 |
| 160 | 0.064 | >8 | 64 |
| 80 | 0.064 | >8 | 64 |
| 40 | 0.064 | >8 | 64 |
| 20 | 0.064 | >8 | 64 |
| 10 | 0.064 | >8 | 64 |
| 5 | 0.064 | >8 | 64 |
| 2.5 | 0.064 | 0.064 | 0.064 |
| 1.25 | 0.064 | 0.064 | 0.064 |
| 0.625 | 0.064 | 0.064 | 0.064 |
| 0.3125 | 0.064 | 0.064 | 0.064 |

^{A}All concentration values are given in μg/ml. IFU, inclusion forming units of Chlamydia; MIC, minimal inhibitory concentration determined as one 2-fold dilution concentration more than the MIC₉₀ (dilution in which 90% or more of inclusions are altered in size and morphology); MCC, minimal chlamydicidal concentration following one freeze-thaw passage in microtiter plates; MCC₃ minimal chlamydicidal concentration following three passages in shell vials.

### Example 3

### Anti-Bacterial Susceptibility Testing of Reference Strains of Chlamydia Species and Clinical Isolates of C. trachomatis

Anti-bacterial susceptibility testing using both reference strains of chlamydiae species and clinical isolates of C. trachomatis was performed using doxycycline, azithromycin, and ofloxacin. The results of in vitro susceptibility testing for each anti-bacterial provided comparable MIC values for the C. trachomatis, C. pneumoniae, and C. psittaci reference strains tested (Table 3). Using the tetracycline-resistant C. suis strain, the MIC values were considerably higher for doxycycline, but were comparable to the other reference strains for azithromycin and ofloxacin. Susceptibility testing of the different clinical isolates of C. trachomatis showed only slight variation of MIC values (by one 2-fold dilution) for doxycycline, azithromycin, and ofloxacin (Table 4). The MCC values determined by microtiter plate passage were consistently less than those determined by shell vial passage for both the chlamydiae reference strains and clinical isolates of C. trachomatis. Further, the MCC values determined by shell vial passage were consistently lower than the MCC₃ values. MCC₃ values were consistently >100×MIC levels demonstrating survival of some organisms at high drug concentrations. When chlamydiae strains with the highest MCC₃ values were retested, there was no homotypic elevation of MIC values, i.e., a pattern in which the majority of organisms survive anti-bacterial concentrations well above the MIC, but rather a repeat of the heterotypic survival, i.e., a pattern in which small numbers of organisms survive well above the MIC, of a small number of organisms at higher MIC concentrations of a given anti-bacterial. For some strains, the retesting of a small number of organisms with the highest MCC₃ values was repeated up to five times, still with no apparent elevation of MIC in this select group of organisms. When MCC₃ values were determined for isolates in different cell lines, those cultured in McCoy and BGMK cells had comparable values. Those tested in HeLa cells demonstrated elevated (>10×MIC) MCC₃ values, but never to the higher degree seen in McCoy and BGMK cell lines, exemplifying the degree of infectivity different cell lines have for chlamydiae. C. pneumoniae strains were found to reach the MCC₃ values of McCoy cell lines when HEp-2 cell lines were used.

The results described herein demonstrates the significant differences between MIC and MCC₃ values (>100×) for the three anti-bacterial agents screened. For each strain there were organisms that were capable of surviving concentrations of anti-bacterial agents that were 100-fold greater than the MIC for each anti-bacterial agent. However, these surviving organisms did not exhibit the features common to development of anti-bacterial resistance. Instead, they demonstrated a heterotypic type of survival, which is more suggestive of the persistent forms of infection. These results demonstrate that for all of the strains tested, a small population of the bacteria was able to persist despite the addition of high levels of three different antibiotics. These results further underscore the need for treatment regimens that can eradicate even these persistent forms of the intracellular bacteria.

TABLE 3
ANTI-BACTERIAL SUSCEPTIBILITIES OF REFERENCE STRAINS OF CHLAMYDIA SPECIES.^{A}

| | Doxycycline | | | | Azithromycin | | | | Ofloxacin | | | |
|---|---|---|---|---|---|---|---|---|---|---|---|---|
| Chlamydia | microtiter | | shell vial | | microtiter | | shell vial | | microtiter | | shell vial | |
| (reference strains) | MIC | MCC | MCC₁ | MCC₃ | MIC | MCC | MCC₁ | MCC₃ | MIC | MCC | MCC₁ | MCC₃ |
| C. trachomatis | 0.064 | 0.125 | >8 | 16-32 | 0.125 | 0.25 | >8 | 64 | 0.5 | 1.0 | >8 | >128 |
| B,D,E,F,G,H,I,Ia,J,K | | | | | | | | | | | | |
| (human genital serovars) | | | | | | | | | | | | |
| C. trachomatis | 0.064 | 0.125 | >8 | 32 | 0.125 | 0.25 | >8 | 64 | 0.5 | 1.0 | >8 | >128 |
| L2 (LGV serovar) | | | | | | | | | | | | |
| C. trachomatis | 0.064 | 0.5 | >8 | 64 | 0.125 | 0.5 | >8 | 128 | 0.5 | 1.0 | >8 | >128 |
| MoPn(mouse pneumonitis strain) | | | | | | | | | | | | |
| C. suis | 1.0 | 2.0 | >8 | >128 | 0.125 | 0.25 | >8 | >128 | 0.5 | 1.0 | >8 | >128 |
| R-19 (porcine strain)^{B} | | | | | | | | | | | | |
| C. pneumoniae^{C} | 0.064 | 0.125 | 4-8 | 32-64 | 0.125 | 0.25 | 4-8 | 64 | 0.5 | 1.0 | >8 | >128 |
| TW-183, CWL-029 | | | | | | | | | | | | |
| C. psittaci | 0.064 | 0.250 | >8 | >128 | 0.125 | 0.50 | >8 | >128 | 0.5 | 2.0 | >8 | >128 |
| 6BC, GPIC | | | | | | | | | | | | |
| ^{A}All concentration values are given in μg/ml. MIC, minimal inhibitory concentration determined as one 2-fold dilution concentration more that the MIC₉₀ (dilution in which 90% or more of inclusions are altered in size and morphology); MCC, minimal chlamydicidal concentration following one freeze-thaw passage in | | | | | | | | | | | | |
| # microtiter plates; MCC₁, minimal chlamydicidal concentration following one passage in shell vials; MCC₃ minimal chlamydicidal concentration following three passages in shell vials. | | | | | | | | | | | | |

^{B}Tetracycline-resistant (MIC > 4.0 μg/ml) porcine strain provided by Arthur A. Andersen, National Animal Disease Center, Ames, IA.
^{C}C. pneumoniae strains grown in HEp-2 cells.

TABLE 4
ANTI-BACTERIAL SUSCEPTIBILITIES OF CLINICAL ISOLATES OF CHLAMYDIA TRACHOMATIS.^{A}

| | Doxycycline | | | | Azithromycin | | | | Ofloxacin | | | |
|---|---|---|---|---|---|---|---|---|---|---|---|---|
| C. trachomatis | microtiter | | shell vial | | microtiter | | shell vial | | microtiter | | shell vial | |
| (clinical isolates) | MIC | MCC | MCC | MCC₃ | MIC | MCC | MCC | MCC₃ | MIC | MCC | MCC | MCC₃ |
| Single isolates | 0.064-0.125 | 0.125-0.25 | >8 | 16-64 | 0.125-0.25 | 0.25-0.5 | >8 | 64 | 0.5-1.0 | 1-2 | >8 | >128 |
| (n = 30) | | | | | | | | | | | | |
| Treatment failure | 0.064-0.125 | 0.125-0.25 | >8 | 16-64 | 0.125-0.25 | 0.25-0.5 | >8 | 64 | 0.5-1.0 | 1-2 | >8 | >128 |
| isolates (n = 6) | | | | | | | | | | | | |
| Recurrent same | 0.064-0.125 | 0.125-0.25 | >8 | 16-64 | 0.125-0.25 | 0.25-0.5 | >8 | 64 | 0.5-1.0 | 1-2 | >8 | >128 |
| serovar isolates | | | | | | | | | | | | |
| (n = 6) | | | | | | | | | | | | |
| CDC strains^{B} | 0.064-0.125 | 0.125-0.25 | >8 | 16-64 | 0.125-0.25 | 0.25-0.5 | >8 | 64 | 0.5-1.0 | 1-2 | >8 | >128 |
| treatment failures | | | | | | | | | | | | |
| (n = 2) | | | | | | | | | | | | |
| CDC strains^{C} | 0.064-0.125 | 0.125-0.25 | >8 | 16-64 | 0.125-0.25 | 0.25-0.5 | >8 | 64 | 0.5-1.0 | 1-2 | >8 | >128 |
| sensitive controls | | | | | | | | | | | | |
| (n = 2) | | | | | | | | | | | | |
| ^{A}All concentration values are given in μg/ml. MIC, minimal inhibitory concentration determined as one 2-fold dilution concentration more that the MIC₉₀ (dilution in which 90% or more of inclusions are altered in size and morphology); MCC, minimal chlamydicidal concentration following one freeze-thaw passage in microtiter plates; | | | | | | | | | | | | |
| # MCC₁, minimal chlamydicidal concentration following one passage in shell vials; MCC₃ minimal chlamydicidal concentration following three passages in shell vials. | | | | | | | | | | | | |

^{B}Strains from 2 treatment failures provided by the CDC and determined to be multi-drug resistant at CDC Laboratories.
^{C}Strains from 2 controls provided by the CDC and determined to be sensitive isolates at CDC Laboratories.

### Example 4

### Comparison of MIC, MCC, MCC₃ and MCC₃/MIC Ratio Utilizing C. trachomatis Serovar D Strain in MCCOY Cells

MIC and MCC determinations for seven different anti-bacterial agents (doxycycline, azithromycin, ofloxacin, tetracycline, erythromycin, rifampin, and rifalazil (KRM-1648)) were compared in order to determine the relative efficacy of each agent in treating a persistent Chlamydia infection. Table 5 summarizes the MIC and MCC determinations for the seven anti-bacterial agents. The MCC for all drugs except rifalazil was many fold higher than the MIC, indicating chlamydial survival in concentrations of anti-bacterial well beyond the MIC for most drugs. When organisms from the highest surviving MCC₃ for each drug were retested, there was no evidence of homotypic elevation in MIC levels but rather a repeated pattern of heterotypic survival of a small number of organisms at high concentrations of drug. These results are consistent with heterotypic survival. Compared with other drugs, rifalazil, an experimental anti-chlamydial compound, was highly active and appeared bactericidal at concentrations much closer to the MIC level. For all of the compounds except rifalazil, the MCC₃/MIC ratio was greater than 128. These results demonstrate the variation in efficacy of anti-bacterial agents in the treatment of persistent infections. These results further demonstrate the use of MCC₃/MIC ratio to quantitatively assess the therapeutic potential of any anti-bacterial for the treatment of persistent intracellular infections.

TABLE 5
COMPARISON OF MIC, MCC, MCC₃, AND MCC₃/MIC
RATIO UTILIZING C. TRACHOMATIS SEROVAR D STRAIN
IN MCCOY CELLS.^{A}

| Anti-bacterial | MIC | MCC | MCC₃ | MCC₃/MIC |
|---|---|---|---|---|
| Doxycycline | 0.064 | 8 | 64 | 1000 |
| Azithromycin | 0.125 | 8 | 64 | 512 |
| Ofloxacin | 1.0 | 16 | 128 | 128 |
| Tetracycline | 0.25 | 16 | 128 | 128 |
| Erythromycin | 0.5 | 8 | 128 | 256 |
| Rifampin | 0.016 | 0.5 | 4 | 250 |
| Rifalazil | 0.00025 | 0.002 | 0.004 | 16 |

^{A}Inhibitory concentrations given in μg/ml.

### Example 5

### Comparison of MIC, MCC, MCC₃ and MCC₃/MIC Ratio Utilizing Clinical Isolates of C. trachomatis

MIC and MCC determinations for five different anti-bacterials (rifalazil, KRM-1657, doxycycline, azithromycin, ofloxacin) were compared in order to determine the relative efficacy of each microbial in treating a persistent Chlamydia infection. Table 6 summarizes the MIC and MCC determinations for the five anti-bacterial agents using clinical isolates of C. trachomatis. The MCC₃ for all drugs except rifalazil and KRM-1657 was many fold higher than the MIC indicating chlamydial survival in concentrations of anti-bacterial well beyond the MIC for most drugs. Rifalazil and KRM-1657 are experimental anti-chlamydial compounds, which were highly active and appeared bactericidal at concentrations much closer to the MIC level.

These results demonstrate the range of efficacy of five different anti-bacterial agents in the treatment of persistent infections and further they demonstrate the use of the MCC₃/MIC ratio to quantitatively assess the therapeutic potential of any anti-bacterial for the treatment of persistent intracellular infections. The use of this method to distinguish between anti-bacterial agents that are effective at eradicating acute infections versus those effective at eradicating chronic infections, allows clinicians to select the appropriate anti-bacterial agent, such as rifalazil in the case of these particular clinical isolates, for treatment.

TABLE 6
IN VITRO DRUG SUSCEPTIBILITIES ON CLINICAL ISOLATES
OF C. TRACHOMATIS^{A}

| Drug | MIC | MCC₁ | MCC₃ | MCC₃/MIC |
|---|---|---|---|---|
| Rifalazil | 0.00025-0.005 | 0.001-0.002 | 0.004-0.008 | 16 |
| KRM-1657 | 0.000064-0.000125 | 0.0005-0.001 | 0.001-0.002 | 16 |
| Doxycycline | 0.064-0.125 | 4-8 | 32-64 | 1000 |
| Azithromycin | 0.125-0.250 | 4-8 | 32-64 | 512 |
| Ofloxacin | 1.0-2.0 | 8-16 | >128 | 128 |
| ^{A}All concentration values are given in μg/ml. MIC, minimal inhibitory concentration determined as one 2-fold dilution concentration more that the MIC₉₀ (dilution in which 90% or more of inclusions are altered in size and morphology); MCC₁, minimal chlamydicidal concentration following one passage in shell vials; MCC₃ minimal chlamydicidal concentration following three passages in shell vials. MIC, MCC₁ values determined for 12 strains (3cx, 3male urth, 3 male | | | | |
| # rectal, and 3PID patients); MCC₃ determined for 6 strains (3cx, 3 male urth). | | | | |

While the preferred embodiment of the invention has been illustrated and described, it will be appreciated that various changes can be made therein without departing from the spirit and scope of the invention.

What is claimed is:

1. A method of screening an anti-bacterial agent for its effectiveness in treating persistent intracellular infections by a bacteria capable of forming intracytoplasmic inclusions in cells, said method comprising the steps of:
(a) inoculating a first culture of cells with said bacteria and then contacting said first culture of cells with said anti-bacterial agent for 24 to 72 hours;
(b) determining the number of intracytoplasmic inclusions that are altered in size and morphology in said contacted first culture of cells, relative to a culture of cells inoculated with said bacteria but not contacted with said anti-bacterial agent;
(c) using the number of altered inclusions determined in step (b) to calculate the minimal inhibitory concentration (MIC) of said anti-bacterial agent;
(d) inoculating a second culture of said cells with said bacteria and then contacting said second culture of cells with an anti-bacterial agent for 24 to 72 hours;
(e) passaging said second culture of cells three times;
(f) determining the number of morphologically normal intracytoplasmic inclusions in said passaged second culture of said cells;
(g) using the number of morphologically normal intracytoplasmic inclusions determined in step (f) to calculate the minimal chlamydicidal concentration after three passages (MCC₃) of said anti-bacterial agent; and
(h) determining the MCC₃/MIC ratio for said anti-bacterial agent, wherein an anti-bacterial agent having a ratio of 100 or less is an anti-bacterial agent that is effective for treating persistent intracellular infections by a bacteria capable of forming intracytoplasmic inclusions in cells.

2. The method of claim 1, wherein said first and second cultures of cells are cultures of McCoy cells and said intracellular bacteria are Chlamydia trachomatis.

3. The method of claim 1, wherein said first and second cultures of cells are cultures of McCoy cells and said intracellular bacteria are Chlamydia psittaci.

4. The method of claim 1, wherein said first and second cultures of cells are cultures of HEp-2 cells and said intracellular bacteria are Chlamydia pneumoniae.

5. The method of claim 1, wherein said first and second cultures of cells are inoculated at a multiplicity of infection of 0.2-2.0 IFU/cell.

6. The method of claim 1, wherein said determining steps (b) or (f) employ immunofluorescence detection.

7. The method of claim 6, wherein said immunofluorescence detection employs a genus-specific antibody.

8. The method of claim 7, wherein said genus-specific antibody is CF-2.

9. The method of claim 1, wherein said contacting of step (a) or step (d) of said inoculated first or second culture of cells with said anti-bacterial agent occurs within 0 to 8 hours of inoculation with said bacteria.

10. The method of claim 1, wherein said first and second cultures of cells comprise HeLa, McCoy, BGMK, Hep-2, HL, or Vero cells.

11. The method of claim 1, wherein said bacteria comprise C. trachomatis, C. pneumoniae, C. psittaci, C. suis, Simkania negevensis, or Parachlamydia acanthamoeba.

12. The method of claim 1, wherein said passaging comprises the steps of:
(a) culturing said second culture of cells in glass shell vials;
(b) sonicating the cultured cells to release intra-cellular inclusions; and
(c) centrifuging the sonicated cells to yield a pellet of cellular material, and a supernatant comprising liberated intracellular inclusions.

13. The method of claim 12 wherein the cultured cells are centrifuged at a speed in the range of from 200×g to 500×g for from 5 minutes to 15 minutes at a temperature of from 1° C. to 11° C.

14. The method of claim 13 wherein the cultured cells are centrifuged at about 300×g, followed by 10 minutes at about 4° C.
